(19) United States
(12) Patent Application Publication
(10) Pub. No.: US 2025/0339622 A1
(43) Pub. Date: Nov. 6, 2025

(54) System for long-term storage of a pharmaceutical composition

(71) Applicant: SCHOTT Pharma Schweiz AG, St. Gallen (CH)

(72) Inventors: Benjamin JÄGER, Stachen (CH); Marcel BERLINGER, Speicher (CH); Christoph STEINLEIN, Romanshorn (CH); Ulrich WALCHER, St. Gallen (CH)

(21) Appl. No.: 18/854,129

(22) PCT Filed: Apr. 5, 2023
(86) PCT No.: PCT/EP2023/058920

§ 371 Date: Oct. 4, 2024

(30) Foreign Application Priority Data
Apr. 7, 2022 (EP) 22167256.1

(51) Int. Cl. A61M 5/31; A61J 1/14; A61M 5/34

(52) U.S. Cl. CPC A61M 5/3129; A61J 1/1468; A61M 5/343; A61M 5/345; A61M 5/346; A61M 5/348; A61M 2205/0238; A61M 2205/21

(57) ABSTRACT

Disclosed is a system for long-term storage of a pharmaceutical composition particularly comprising a syringe. The system comprises an adapter for fixing a needle to the syringe barrel providing improved container closure integrity.

The present invention relates to a system for long-term storage of a pharmaceutical composition particularly comprising a syringe. The system comprises an adapter for fixing a needle to the syringe barrel providing improved container closure integrity.

### BACKGROUND

Prefilled syringes are commonly used as a long-term storage system of pharmaceutical compositions in a ready-to-use state. The pharmaceutical composition is filled into the syringe which is already equipped with a needle and a protective cap, also known as the needle shield in the relevant art. The needle shield usually serves several purposes at once, namely to protect the person handling the syringe from injury, to protect the needle and particularly the needle bevel from damage, and to ascertain sterility of the pharmaceutical composition within the syringe and of the needle until use.

### SUMMARY OF THE INVENTION

Different connectors exist for fixing the needle to the syringe barrel. However, the existing connectors have proven themselves to be only partially suitable for long-term storage of the prefilled syringes with staked needles, in particular at very low or elevated temperatures (e.g. −80° C. like required by some vaccines or 40° C. and high humidity for tropical climate). There are clear deficiencies in the container closure integrity, i.e. the content of the filled syringe may leak and/or become contaminated.

An object of the present invention is, hence, to overcome the disadvantages of the prior art. In particular, the long-term storage capabilities shall be improved in terms of container closure integrity.

It is an object of the present invention to provide a system for long-term storage of a pharmaceutical composition

In a first aspect, the invention relates to a system for long-term storage of a pharmaceutical composition, comprising:
a syringe barrel, comprising:
   a front end comprising a cone, and
   a back end;
a plunger inserted into the back end; and
an adapter connected to the front end, comprising:
   a needle,
   an adapter body connecting the needle with the syringe barrel, and
   optionally a needle shield covering the needle.

In a second aspect, the invention relates to a system for long-term storage of a pharmaceutical composition, comprising:
a syringe barrel, comprising:
   a front end comprising a cone, and
   a back end;
a plunger inserted into the back end; and
an adapter connected to the front end, comprising:
   a needle,
   an adapter body connecting the needle with the syringe barrel, and
   optionally a needle shield covering the needle;
      wherein a pull-off force of the adapter is 1 N to 500 N, measured according to ISO 11040-4:2015, Annex G.3.

In a third aspect, the invention relates to a system for long-term storage of a pharmaceutical composition, comprising:
a syringe barrel, comprising:
   a front end comprising a cone, and
   a back end;
a plunger inserted into the back end; and
an adapter connected to the front end, comprising:
   a needle,
   an adapter body connecting the needle with the syringe barrel, and
   optionally a needle shield covering the needle;
      wherein a cone breakage force is 1 N or more and/or 400 N or less, measured according to ISO 11040-4:2015, Annex C.2.

In a fourth aspect, the invention relates to a system for long-term storage of a pharmaceutical composition, comprising:
a syringe barrel, comprising:
   a front end comprising a cone, and
   a back end;
a plunger inserted into the back end; and
an adapter connected to the front end, comprising:
   a needle,
   an adapter body connecting the needle with the syringe barrel, and
   optionally a needle shield covering the needle;
      wherein a pull-off force of the adapter is 1 N to 500 N, measured according to ISO 11040-4:2015, Annex G.3, and a cone breakage force is 1 N or more and/or 400 N or less, measured according to ISO 11040-4:2015, Annex C.2.

One of the connectors used for attaching the needle to the syringe is the Luer lock connector comprising a cone on the barrel for receiving a respective receiving inner cone on the needle part. Both parts are fixed by means of an adapter. This adapter snaps over an undercut of the cone and locks the needle in place. The inventors have discovered that during this snapping action, the surface of the cone and/or the undercut area may be damaged by the retaining part with scratches or impact stress. This may lead to leakage during long-term storage or, in the worst case, to a breakage of the cone resulting in reduced container closure integrity.

However, on the other hand the locking part has to sit sufficiently tight on the cone in order to provide a certain minimum pull-off force of the needle and adapter for ascertaining that the needle assembly is not accidentally removed when handling the prefilled syringe or pulling off the needle shield. In addition to this, the pressure exerted by the adapter also improves the container closure integrity by better sealing the contacting surfaces, in particular when used in combination with a resilient sealing member.

In some optional variants, the pull-off force of the adapter may be 50 N to 400 N, preferably 80 N to 350 N, more preferably 100 N to 300 N, more preferably 120 N to 250 N, more preferably 140 N to 200 N, measured according to ISO 11040-4:2015, Annex G.3 and/or the cone breakage force may be 5 N or more, preferably 20 N or more, more preferably 40 N or more, more preferably 50 N or more, more preferably 60 N or more and/or 300 N or less, preferably 200 N or less, more preferably 150 N or less, measured according to ISO 11040-4:2015, Annex C.2. The pull-off force of the adapter may be at least 50 N, at least 60 N, at least 70 N, at least 80 N, or at least 85 N. The pull-off force of the adapter may be at most 50 N to 400 N, at most 300 N, at most 250 N, at most 200 N, or at most 150 N. The cone breakage force may be at least 5 N, at least 20 N, at least 40 N, at least 50 N, or at least 60 N. The cone breakage force may be at most 300 N, at most 200 N, or at most 150 N. The cone breakage force may be 5 N to 300 N, or 20 N to 200 N, or 40 N to 200 N, or 50 N to 150 N, or 60 N to 150 N.

In preferred embodiments, the system passes the container closure integrity test according to ISO 11040-4:2015, Annex H. In this test, the syringe is filled with liquid and submerged in a dye solution. The syringe is then visually inspected for ingression of dye solution after a depressurization/re-pressurization cycle. The system according to this disclosure passes this test conducted with fresh filled samples.

In optional embodiments, the system passes the container closure integrity test according to ISO 11040-4:2015, Annex H after a storage time of 7 days, preferably 30 days, more preferably 100 days, more preferably 3 months, more preferably 6 months, more preferably 1 year, more preferably 2 years, more preferably 5 years at 15° C.-30° C. at ambient conditions or at 40° C.±2° C. at 75±5% relative humidity. This proves the superior long-term storage properties of the system. The filled syringes may be stored for extended time before testing at room temperature and ambient conditions or even at the elevated temperature of 40° C. and high humidity based on the conditions for accelerated aging tests as mentioned in the ICH guidelines ICH Q1A (“Stability Testing of New Drug Substances and Products”) and still pass the dye test.

In some embodiments of the system, the syringe barrel comprises, or is made of, glass; and/or the adapter body comprises polymer.

In further embodiments, the syringe barrel comprises a shoulder and the cone comprises a tapering region including the cone's broadest outer circumference, and an undercut having an outer circumference smaller than the cone's broadest outer circumference, wherein preferably the undercut is located between the tapering region and the shoulder of the syringe barrel.

In some embodiments, the adapter may have an adapter rotation resistance force on the cone of 0.03 Nm to 1 Nm, preferably 0.04 Nm-0.6 Nm, preferably 0.05 Nm-0.4 Nm, preferably 0.06 Nm-0.3 Nm. The adapter rotation resistance force may be determined based on ISO 11040-4:2015, Annex G.4. The adapter may have an adapter rotation resistance force on the cone of at least 0.03 Nm, at least 0.04 Nm, at least 0.05 Nm, or at least 0.06 Nm. The adapter may have an adapter rotation resistance force on the cone of at most 1 Nm, at most 0.6 Nm, at most 0.4 Nm, or at most 0.06 Nm-0.3 Nm. The adapter rotation resistance force is referring to the force which is required to rotate the adapter in its assembled state on the cone of the syringe around the longitudinal central axis of the syringe barrel. Hence, the connection is torque-proof up to the indicated force level. This value is indicative of the tightness of the connection between the adapter and the cone. Hence, it should not be too low.

In further embodiments, the adapter is tilt-proof fitted to the syringe barrel so that a central axis of the needle is congruent with a central axis of the syringe barrel. This means that the adapter is fitted to the syringe barrel with sufficiently restricted possibility of lateral tilt so that the needle remains in the central axis of the syringe barrel. This is particularly important for avoiding damage to the needle bevel when putting the needle shield on the syringe.

In embodiments, the needle is mounted fixed or movable within the adapter body. In its simplest form, the needle is mounted fixed in the adapter body. It can also be designed to be movable along the longitudinal axis of the adapter by this keeping the orientation of the needle in line with the syringe barrel. This design allows for the construction of single use syringes which are capable of retracting the needle in order to prevent a second use. Both options are suitable for the function of the adapter design of the present disclosure.

The adapter body may comprise a first part supporting the needle, and a second part being in contact with the cone, preferably with the undercut of the cone. The adapter body is, hence, not a single work piece but assembled from two separate parts whereof one holds the needle and the second one establishes the connection to the syringe barrel.

In some embodiments, the first part and the second part are irreversibly connected, preferably by a click mechanism. In particular, the connection is not exclusively made by a screwing connection. A click mechanism is referring to a connection which is established by means of a form fit which engages in a snapping action and locks the parts. Thus, a blocking of the translatory movement is generated in the direction of force of the system's axis of rotation.

Optionally, the setting force of the second part of the adapter on the cone and/or the setting force of the adapter on the cone and/or the force to irreversibly connect the first part and the second part by a click mechanism to reach the click point of the click mechanism is 10 N to 300 N, preferably 20 N to 150 N, more preferably 50 N to 120 N. The setting force for the adapter to reach the click point of the click mechanism may be at least 10 N, at least 20 N, or at least 50 N. The setting force for the adapter to reach the click point of the click mechanism may be at most 300 N, at most 150 N, or at most 120 N. The setting force to reach the click point is the force which is required for pressing the parts together until they snap together and lock. These values may be predefined on the setting machine. By using a way controlled system the forces need to be in the defined range. By using a force controlled system the forces are to be adjusted within the given range in order to fulfill the setting process. The setting process is fulfilled when the second part is pushed to the undercut of the cone. The assembly process may either comprise to first assemble the first and second part and thereafter the whole adapter with the syringe barrel or to first assemble the second part with the syringe barrel and thereafter the first part with the already mounted second part.

In embodiments, the material of the second part comprises or consists of a polymer.

When pushing the second part over the cone of the syringe barrel for locking the adapter on the syringe, the polymer material of the second part may be chosen such that the deformation of the second part is elastic or elastic and plastic. In some useful embodiments, the polymer material of the second part is chosen such that the deformation of the second part is elastic and plastic.

In optional embodiments, the polymer of the second part may be chosen from polypropylene (PP), polyethylene terephthalate (PET), or polyamide (PA) as well as their copolymers and blends containing at least 50 wt-% of said polymers. Optionally, the material is chosen from polypropylene (PP) or polyamide (PA), such as PA 11. Particularly useful is polypropylene.

To achieve the goal of a fixation of the needle adapter on the syringe without damaging the material of the syringe barrel, in particular when it is made of glass, one suitable measure contributing to this may be to choose the material of the second part, in particular the type of polymer, such that a ratio of the Vickers hardness of the syringe barrel material to the shore D hardness of the material of the second part is larger than 5.86. Optionally the ratio is larger than 5.86, larger than 6.44, or larger than 7.25. The ratio may be from >5.86 to 10, from >6.44 to 9.5, or from >7.25 to 9. The ratio may be at most 10, at most 9.5, or at most 9. The shore hardness may be determined according to DIN ISO 7619-1:2012-02. The Vickers hardness may be determined according to DIN EN ISO 6507-1:2018-07.

In some embodiments, the second part, whose material comprises or consists of a polymer, comprises a retaining structure element essentially having a closed ring shape which exerts a spring force in a direction of its central axis. The central axis is referring here to the axis perpendicular to the diameter of the ring shape. When assembled on the syringe, this spring force acts along the central axis of the syringe barrel on the undercut of the cone and pulls the adapter elastically towards the cone. This can improve the container closure integrity, in particular when used at very low or high temperatures. The ring shape may also include elements which are arranged out of plane, i.e. non-perpendicular to the diameter of the ring shape. The entire ring shape may also be non-planar, such as for example a frustoconical surface.

Optionally, the polymer of the second part is stretchable, i.e. it has a relatively high yield strength and a residual strain range remains after passing through the deformation range. Hence, polypropylene is a suitable material whereas, for example, COC may in some embodiments not be ideal because while it has a very high yield strength, it breaks at high stress. Polyamide has a lower residual strain range than polycarbonate and is, hence, not the best choice for some embodiments.

The second part may particularly be designed to be deformed elastically and plastically. A main object of the design and material choice of the second part for fixing the adapter tightly on the cone of the syringe barrel consists in a tolerance compensation between the surfaces of the undercut of the cone and the second part. A standard Luer lock geometry of a 1 ml syringe, for example, may have according to ISO 594-1:1986 a tolerance of the outer diameter of the undercut of 4.34 mm (+0.00 mm/−0.14 mm) and of the largest outer diameter of the cone of 4.43 mm±0.07 mm. The second part can be made in a shape and with a material that is capable of compensating these tolerances including its own diameter tolerance to ascertain a certain pressure on the undercut which is sufficient for the required pull off force for the adapter on the one hand and a tight connection between the cone and its mating surface of the second part and compression of a sealing member on the other hand. This effectively increases the container closure integrity.

In embodiments following this design approach, the geometric design of the second part and/or the polymer for the second part are selected such that the maximal deformation of the second part when being slid over the cone onto the undercut of the syringe results in a strain ε of the polymer within a range of εₑₗ≤ε≤ε_{Y} or a range of εₑₗ≤ε≤εₘₐₓ for polymers without a yield point, wherein εₑₗ is the strain at the elastic limit, ε_{Y} is the strain at the yield point and εₘₐₓ is the maximum strain, and/or the second part has at a position on the undercut of the cone a residual stress σᵣₑₛ from a remaining elastic deformation εᵣₑₛ.

The elastic limit is defined here as the maximum stress that can be applied at a temperature of 23° C. without resulting in permanent deformation when unloaded.

The yield point is defined here as the stress at a temperature of 23° C. at which plastic flow (yielding) begins and there are large increases in strain with little or no increase in stress as shown in ISO 527-1:2019.

In the setting process, the second part is widened by the cone. In a stress-strain-diagram, the widening follows the curve through the elastic section into the elastic-plastic section up to a maximum of the yield point. By the material choice, its deformation does not exceed the yield strain before snapping back onto the undercut. Thus, the final widening Δd of the second part on the undercut of the Luer lock of the syringe will be in the range of

$$Δ {d}_{min} > 0$$

$$Δ {d}_{max} = {ε}_{Y} {d}_{0} - 2 {h}_{undercut} + tol < {ε}_{Y} {d}_{0}$$

wherein d₀ is the nominal diameter, h_{undercut} is the height of the syringe cone undercut, and tol are the summarized machining tolerances of the second part and the Luer lock adapter on the syringe. While the resulting plastic deformation compensates the tolerances, the residual stress σᵣₑₛ from the remaining elastic deformation εᵣₑₛ on the undercut may act to pull the adapter onto the cone, thereby compressing a sealing member arranged between the tip of the cone and the first part as described above.

For example, the retaining structure element of the second part may be stretched by 20 μm when being slid over the cone and will contract to a remaining stretching by 10 μm when snapping onto the undercut. This remaining plastic stretching will compensate the tolerances in the respective diameters. As a result, the retaining structure element will always have a residual stress on the undercut which ascertains the tight fit.

In preferred embodiments, a sealing member is arranged between the first part and the syringe barrel. The sealing member may be important for the container closure integrity since it seals the connection between the needle and the syringe barrel. By varying the properties of the sealing member, it is possible to optimize the assembling and sealing process. It is particularly advantageous to adjust them in correlation to the intended setting force and adapter holding and rotation resistance forces.

In variants, the sealing member is in contact with the cone, preferably the terminal part of the cone located at the distal side of the syringe barrel. This achieves a very effective sealing and allows for the option of compressing the sealing member.

In further embodiments, the sealing member has a Shore A hardness, measured according to ASTM D2240:2021, 10 seconds, of 20 to 80, preferably 30 to 70, more preferably 45 to 65, more preferably 55 to 60. The sealing member may have a Shore A hardness of at least 20, at least 30, at least 45, or at least 55. The sealing member may have a Shore A hardness of at most 80, at most 70, at most 65, or at most 60. This range has been found to be optimal for the sealing properties and the compression properties.

In some embodiments, the sealing member is compressed by the click mechanism at least partially, preferably at least partially by 10% to 80%, preferably 20% to 70%, more preferably 30% to 60%, more preferably 40% to 50%. The sealing member may be compressed by the click mechanism at least by 10%, at least by 20%, at least by 30%, or at least by 40%. The sealing member may be compressed by the click mechanism at most by 80%, at most 70%, at most 60%, or at most 50%. This can achieve good results in terms of the sealing and the stability and integrity of the connection between the adapter and the syringe barrel. The mechanical compression behavior can be determined via non-linear Finite-Element simulation. The material model reproduces the non-linear stress-strain behavior of the material, differentiating in uni-axial and multi-axial loading. The simulation model consists of solid elements with at least four integration points per element. The FE mesh features minimum 50 elements over thickness of the body. In the simulation, the cone is pressed onto the sealing member towards the level defined by the technical design. The maximum resulting true-strain of the sealing member is measured.

In further embodiments, a Young's modulus of the sealing member is from 0.1 MPa to 5 MPa, preferably from 1 MPa to 4 MPa, more preferably from 1.5 MPa to 3 MPa, determined according to ISO 527-1/-2:2019. The Young's modulus of the sealing member may be at least 0.1 MPa, at least 1 MPa, or at least 1.5 MPa. The Young's modulus of the sealing member may be at most 5 MPa, at most 4 MPa, or at most 3 MPa.

The Young's modulus can be determined with a test setup according to ISO 527-1/-2:2019. The specimen geometry 5A or 5B may be used. In addition, a 3D camera system (for example GOM ARAMIS 12M) can be used in order to measure local surface strain via digital image correlation (DIC). At least 100 images of the ongoing test must be recorded. End of the test is failure of the specimen. True strain/Hencky strain (ε_{true,lateral}) is measured. In DIC, minimum 100 overlapping facets are necessary over the width of the specimen. The force is measured by the material testing machine (load cell ≤5 kN). Strain information of the DIC must lie on the same time axis as the force signal. Lateral strain is assumed to be equal in both lateral directions. True stress is calculated by the formula:

$$\frac{Force}{Sectional Area} \cdot exp ( - 2 \cdot {ε}_{true , lateral} )$$

Young's modulus is determined as the initial slope in the stress-strain diagram.

In embodiments, a thickness of the sealing member, preferably in its compressed state, is 0.05 mm to 3.00 mm, preferably 0.5 mm to 2.50 mm, preferably 0.80 mm to 2.20 mm. The thickness is referring to the dimension of the sealing member which is parallel to the central axes of the needle and the syringe barrel when assembled. The thickness of the sealing member, preferably in its compressed state, may be at least 0.05 mm, at least 0.5 mm, or at least 0.80 mm. The thickness of the sealing member, preferably in its compressed state, may be at most 3.00 mm, at most 2.50 mm, or at most 2.20 mm. The thickness in the uncompressed state may be determined by means of a caliper. The mechanical compression behavior can be determined via non-linear Finite-Element simulation. The material model reproduces the non-linear stress-strain behavior of the material, differentiating in uni-axial and multi-axial loading. The simulation model consists of solid elements with at least four integration points per element. The FE mesh features minimum 50 elements over thickness of the body. In the simulation, the cone is pressed onto the sealing member towards the level defined by the technical design.

In further embodiments, the material of the sealing member comprises, preferably consists of, a polymer, preferably an elastomer, more preferably a thermoplastic elastomer. In particular the thermoplastic elastomers offer the advantage of the moldability by injection molding in combination with elasticity for achieving a good sealing.

In other embodiments, the second part is a retaining part.

In embodiments, the second part has essentially a ring shape which is not fully closed and/or has a gap and/or which can be widened in diameter. This reduces the forces exerted on the cone and the undercut during assembling of the system. An option for easy assembling without damage to the cone or undercut can be the insertion of a wedge member in such a gap which can be removed by means of a lug after sliding the second part over the cone.

In embodiments, the second part has essentially a ring shape which exerts a spring force in a direction of its central axis. The central axis is referring here to the axis perpendicular to the diameter of the ring shape. When assembled on the syringe, this spring force acts along the central axis of the syringe barrel on the undercut of the cone and pulls the adapter elastically towards the cone. This can improve the container closure integrity, in particular when used at very low or high temperatures.

In some embodiments, a ratio of an inner circumference of the second part to the cone's broadest outer circumference is between 85% [mm/mm] and 99% [mm/mm] or between 90% [mm/mm] and 99% [mm/mm], when determined by measuring an inner diameter of the second part by means of a visual measurement device after disassembling it and elastic relaxation. The inner diameter of the second part is measured after its plastic deformation in the assembly process. For measuring, the cone may be broken and the second part may be removed for elastic relaxation. With an optical microscope (for example Optometron UI-1540-C), the inner diameter can be determined.

In some embodiments, a ratio of the inner circumference of the second part to the circumference of the undercut of the cone is from 90% [mm/mm] up to 107% [mm/mm], when determined by measuring the inner diameter of the second part by means of a visual measurement device after disassembling it and elastic relaxation. The inner diameter of the second part is measured after its plastic deformation in the assembly process. For measuring, the cone may be broken and the second part may be removed for elastic relaxation. With an optical microscope (for example Optometron UI-1540-C), the inner diameter can be determined.

In further embodiments, a ratio of a radial force of the second part to the pull-off force of the adapter is 1% to 20,000% [N/N], preferably 2% to 5,000% [N/N], more preferably 5% to 200% [N/N], more preferably 10% to 100% [N/N], more preferably 20% to 50% [N/N]. The ratio may be at least 1% [N/N], at least 2% [N/N], at least 5% [N/N], at least 10% [N/N], or at least 20% [N/N]. The ratio may be at most 20,000% [N/N], at most 5,000% [N/N], at most 200% [N/N], at most 100% [N/N], or at most 50% [N/N]. The radial force is referring to the force exerted by the essentially a ring shaped second part in its radial direction and in the assembled state.

In embodiments, the radial force of the second part is 5 N to 200 N, preferably 10 N to 180 N, more preferably 20 N to 150 N, more preferably 30 N to 120 N, more preferably 40 N to 100 N, more preferably 50 N to 80 N. The radial force of the second part may be at least 5 N, at least 10 N, at least 20 N, at least 30 N, at least 40 N, or at least 50 N. The radial force of the second part may be at most 200 N, at most 180 N, at most 150 N, at most 120 N, at most 100 N, or at most 80 N. For determining the radial force, the second part can be simulated via non-linear Finite-Element simulation. The simulation model consists of solid elements (quads) with at least four integration points per element. The FE mesh features minimum ten elements over thickness of the body. The second part is widened to the max diameter of the syringe barrel cone and afterwards relaxed to the circumference of the undercut. The resulting reaction force is measured in radial direction.

In further embodiments, the pull-off force of the adapter is the pull of force of the second part. This means that the force for attaching the adapter to the syringe barrel is provided only by the second part. There are no other parts required for this purpose.

In embodiments, the material of the second part comprises, preferably consists of, a metal, preferably a metal comprising iron and/or aluminum, more preferably stainless steel. These materials provide good retaining functions and durability and can easily be stamped and pressed into the required shape.

In further embodiments, a thickness of the second part is 0.03 mm to 1 mm, preferably 0.05 mm to 0.8 mm, more preferably 0.1 mm to 0.4 mm, more preferably 0.15 mm to 0.3 mm. The thickness of the second part may be at least 0.03 mm, at least 0.05 mm, at least 0.1 mm, or at least 0.15 mm. The thickness of the second part may be at most 1 mm, at most 0.8 mm at most 0.4 mm, or at most 0.3 mm. The thickness is referring in this case to the thickness of the flat material in the axial direction of the essentially ring-shaped second part, i.e. without considering the external dimensions of the three-dimensionally shaped part. The thickness can be measured via caliper (resolution/precision±0.001 mm). The measurement is repeated at at least five different positions from which the arithmetic mean is calculated.

In embodiments, a ratio of the Young's modulus [GPa], determined according to ISO 527-1/-2:2019, to the thickness of the second part [mm] is 50 to 10,000 [GPa/mm], preferably 100 to 8,000 [GPa/mm], preferably 200 to 5,000 [GPa/mm], preferably 300 to 2,000 [GPa/mm], preferably 500 to 1,000 [GPa/mm]. The ratio may be at least 50 [GPa/mm], at least 100 [GPa/mm], at least 200 [GPa/mm], at least 300 [GPa/mm], or at least 500 [GPa/mm]. The ratio may be at most 10,000 [GPa/mm], at most 8,000 [GPa/mm], at most 5,000 [GPa/mm], at most 2,000 [GPa/mm], or at most 1,000 [GPa/mm]. This ratio has been found to provide the best mechanical properties to the second part in relation to its dimensions and the achieved retaining function.

In some embodiments, the second part is completely surrounded by the first part and/or the second part is embedded in the first part.

In order to decrease the damage of the cone and undercut, the thickness of the lugs of the essentially ring-shaped second part may be set in relation to the width of the lugs measured along the inner circumference. The ratio of the width of the lugs measured along the inner circumference to the thickness of the lugs should be in the range of 1 [mm/mm] to 450 [mm/mm], preferably 2 [mm/mm] to 100 [mm/mm], more preferably 3 [mm/mm] to 50 [mm/mm], more preferably 4 [mm/mm] to 20 [mm/mm] depending on the material used. The ratio may be at least 1 [mm/mm], at least 2 [mm/mm], at least 3 [mm/mm], or at least 4 [mm/mm]. The ratio may be at most 450 [mm/mm], at most 100 [mm/mm], at most 50 [mm/mm], or at most 20 [mm/mm].

Further, the length of the slots in the lugs and the angle of the lugs relative to the central axis of the ring may be varied for optimization. The length of the slots should be in the range of 0 mm to 3 mm, preferably 0.1 mm to 2 mm, more preferably 0.4 mm to 0.8 mm for optimal results. The length may be at least 0 mm, at least 0.1 mm, or at least 0.4 mm. The length may be at most 3 mm, at most 2 mm, or at most 0.8 mm.

In other embodiments, the cone and/or the second part comprise(s) at least one area which is coated by a single-layer or multi-layer coating. Optionally, the cone comprises the coating. A multi-layer coating may comprise multiple layers of the same coating material, i.e. a repeated application of a single material in thinner layers, or layers of different coating materials. The latter is particularly useful for providing different functions to the coating, for example a dampening effect and a scratch resistance effect or surface defect sealing effect. In cases where cone and second part are coated, the different functions may also be applied to them separately. For example, the second part may be provided with a dampening coating and the cone with a scratch resistance and/or surface defect sealing coating.

In embodiments, the at least one area comprises the tapering region having a broadest circumference of the cone and/or the undercut of the cone, preferably the undercut. The tapering region and particularly the undercut are the most critical areas regarding damage and leaking. They can effectively be protected by a suitable coating for improving the long-term container closure integrity.

In embodiments, the coating reduces surface defects on the cone and/or reduces the impact when the second part is clicked to the cone. A suitably coated cone is more resistant to the scratching of the mating second part and dampens the impact of the retaining mechanism snapping onto the undercut. The same applies to a coating on the second part which imparts less scratching surface properties and a dampening effect.

In embodiments, the coating comprises a polymer, in particular an elastomer and/or a thermoplastic and/or a duromer and/or a silicone, and/or a ceramic.

In further embodiments, the coating has a thickness, preferably a mean thickness, of 40 nm to 200 μm, preferably 70 nm to 60 μm, preferably 80 nm to 50 μm, preferably 90 nm to 40 μm. The thickness of the coating may be at least 40 nm, at least 70 nm, at least 80 nm, or at least 90 nm. The thickness of the coating may be at most 200 μm, at most 60 μm, at most 50 μm, or at most 40 μm. The thickness of the coating can be determined by measurement with spectral interferometry (for example Hamamatsu Optical NanoGauge).

In embodiments, a ratio of the pull-off force [N] to a thickness of the coating [nm] is 0.0004 to 8.75 [N/nm], preferably 0.0005 to 4 [N/nm], more preferably 0.0025 to 1 [N/nm], more preferably 0.01 to 0.1 [N/nm]. The ratio may be at least 0.0004 [N/nm], at least 0.0005 [N/nm], at least 0.0025 [N/nm], or at least 0.01 [N/nm]. The ratio may at most 8.75 [N/nm], at most 4 [N/nm], at most 1 [N/nm], or at most 0.1 [N/nm].

In further embodiments, a ratio of the hardness of the glass, expressed as the Young's Modulus in MPa, determined according to ISO 527-1/-2:2019, to the hardness of the coating, expressed as Young's Modulus in MPa, determined according to ISO 527-1/-2:2019, is 4 to 10,000 [MPa/MPa], preferably 10 to 1,000 [MPa/MPa], preferably 20 to 500 [MPa/MPa], preferably 40 to 200 [MPa/MPa], preferably 60 to 100 [MPa/MPa]. The ratio may be at least 4 [MPa/MPa], at least 10 [MPa/MPa], at least 20 [MPa/MPa], at least 40 [MPa/MPa], or at least 60 [MPa/MPa]. The ratio may be at most 10,000 [MPa/MPa], at most 1,000 [MPa/MPa], at most 500 [MPa/MPa], at most 200 [MPa/MPa], or at most 100 [MPa/MPa].

### BRIEF DESCRIPTION OF THE DRAWINGS

FIG. 1 is a cross-sectional view of an adapter body comprising the connected first and second part in its assembled state.

FIG. 2 is a cross-sectional view of an adapter body comprising the connected first and second part in its assembled state including a needle and a sealing member.

FIG. 3 are a top view and a perspective view of a second part having essentially a ring shape with a gap.

FIG. 4a/4b are perspective views of a detail of a ring-shaped second part.

FIG. 5 is a cross-sectional view of a syringe barrel with a cone and a coating thereon.

FIG. 6 is a perspective view of a coated ring-shaped second part.

FIG. 7 is a cross-sectional view of an adapter body comprising the first and second part connected by a click mechanism in its assembled state.

FIG. 8 is a cross-sectional view of an adapter body comprising the first and second part connected by a screw on mechanism in its assembled state.

### DETAILED DESCRIPTION

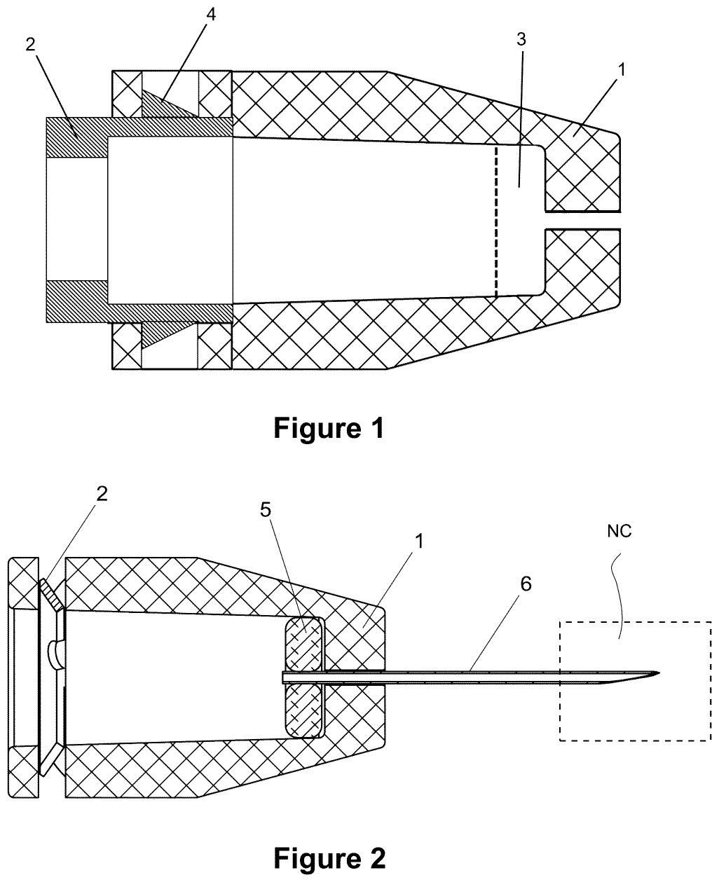

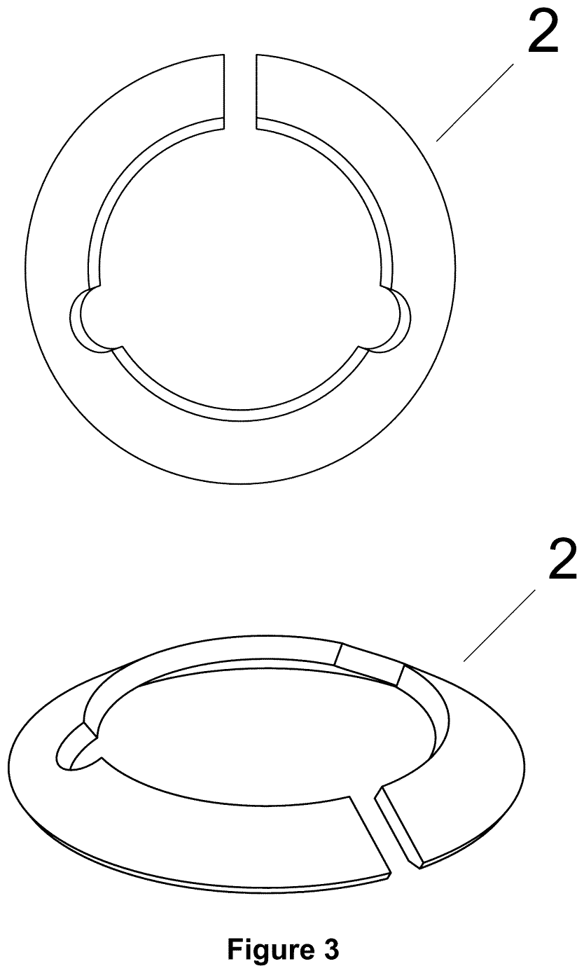

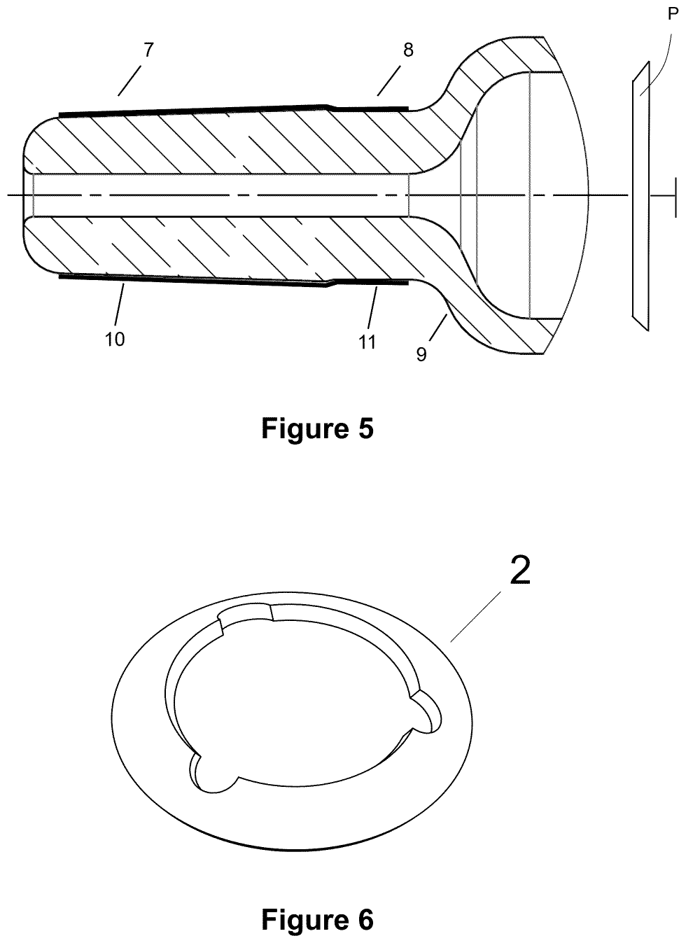

A first example of an adapter body according to the invention is shown in FIG. 1 for attachment for example to the syringe barrel shown in FIG. 5. In a cross-sectional view, there is shown the adapter body comprising the connected first part (1) and second part (2) in its assembled state. Within the first part (1), there is indicated by the dashed line a sealing member zone (3) for receiving a sealing member (5) (see, e.g, FIG. 2). The sealing member (5) serves for sealing the connection between a needle (6) (see, e.g, FIG. 2) that is to be inserted in the first part (1) and the cone of a syringe barrel which is to be inserted through the second part (2). The first part (1) and the second part (2) are snapped together and locked by means of the locking part (4) during assembly of the syringe. The second part (2) is first slid over the cone of the syringe barrel and locks with the smaller diameter aperture on the left side of the figure onto the undercut (8) of the cone (see, e.g. FIG. 5). Then the first part (1) including the needle (6) and sealing member (5) is slid onto the second part (2) and stabilizes the connection to the cone and particularly the undercut (8). The softer material of the second part (2) avoids damage to the cone and undercut (8), while the stiffer first part (1) thereafter ascertains the required stability of the connection. In a variant of the example, both materials may also be the same.

A second example is shown in FIG. 2. In this embodiment, the second part (2) is a retaining ring element which is inserted in the base of the first part (1). The needle (6) is fixed in the upper end of the first part (1) and surrounded by the sealing member (5) within the sealing member zone (3). For assembly, the ring-shaped second part (2) and the sealing member (5) are inserted into the base and tip of the first part (1), respectively, and thereafter the assembled adapter is pushed onto the cone of the syringe barrel. An optional needle cover NC is shown solely schematically.

FIG. 3 shows a top view and a perspective view of a corresponding second part (2) having essentially a ring shape with a gap. This version can be used as is and exerts less force on the cone and undercut. Or it can be dimensioned with its inner diameter more closely to the outer diameter of the undercut (8) and be used together with a wedge member inserted into the gap. After pushing the adapter assembly over the cone to the undercut, the wedge member is removed to tighten the connection.

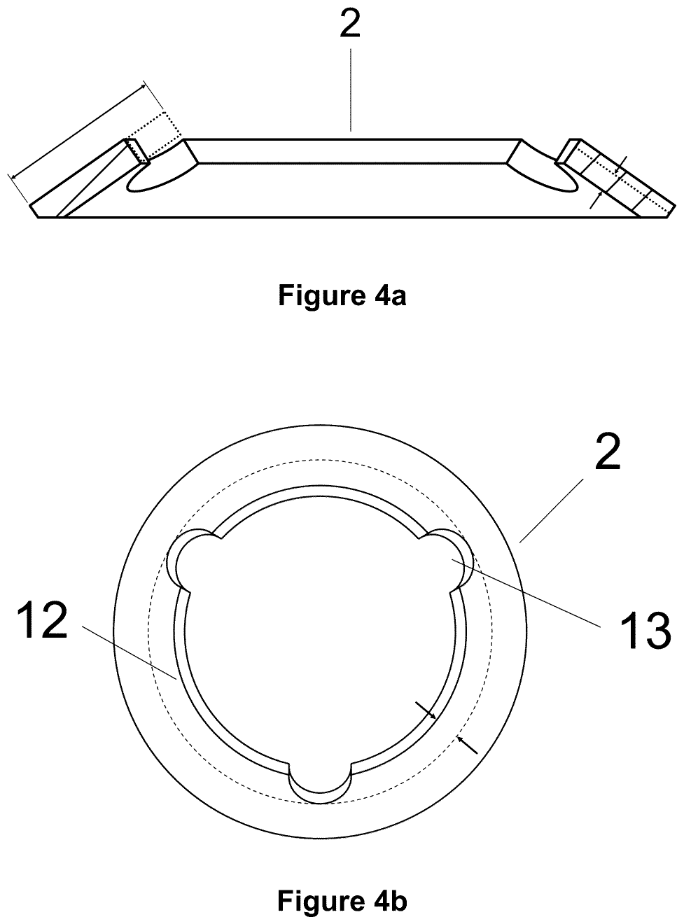

Further variants of the essentially ring-shaped second part (2) are shown in FIGS. 4a and 4b. In order to decrease the damage of the cone and undercut (8), as indicated by the dashed contours and the arrows in the figure, the thickness of the lugs (12) of the essentially ring-shaped second part (2) may be set in relation to the width of the lugs (12) measured along the inner circumference. The ratio of the width of the lugs (12) measured along the inner circumference to the thickness of the lugs (12) in this example is 18 [mm/mm]. As shown in FIG. 4b, the length (indicated by the two arrows and the dashed line) of the slots (13) in the lugs (12) may be varied for optimization. The length of the slots (13) in this shown example are 0.6 mm for optimal results.

A third example is shown in FIG. 5, which is a cross-sectional view of a syringe barrel with a cone and a coating thereon. Shown in detail is only the distal end of the barrel, with a plunger P shown solely schematically at the proximal end. The tip of the syringe barrel is formed by the cone comprising the tapering region (7) and the undercut (8) which then enters into the shoulder (9) region leading to the barrel cylinder. In FIG. 5, there is shown a coating on the tapering region coating area (10) and the undercut coating area (11). The tapering region coating area (10) is coated with a scratch resistant thermoplastic coating and the undercut coating area (11) with a dampening silicone coating.

FIG. 6 shows a perspective view of a coated ring-shaped second part (2). The whole second part (2) is covered with a silicone coating.

### Experimental Results

### Adapters Using a Polymer Material for the Second Part

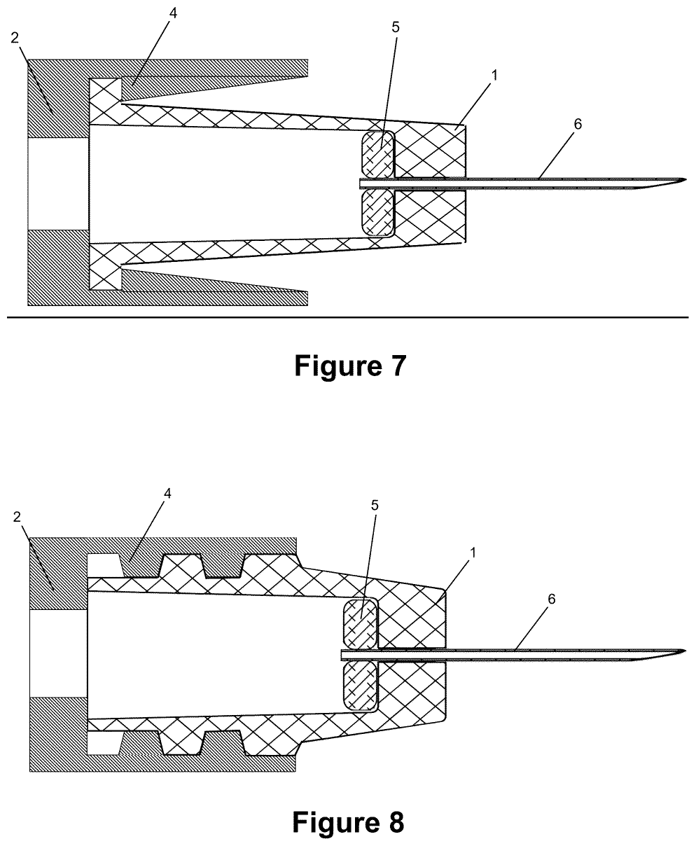

Two different polymer material versions and two different construction principles have been examined regarding their protection performance and properties. The first version is an adapter comprising a second part made of polyamide 11 (PA 11) or polypropylene (PP) using a click mechanism for the connection between the first and the second part. A cross-sectional view of such an adapter is shown in FIG. 7. The design of this adapter is some kind of an inverse form of the adapter design as shown in FIG. 1. In FIG. 1, the first part (1) has a larger diameter than the second part (2) and has the undercut formed in its inside surface for engaging with the slanted region of the second part (2) when it is pushed into it. In FIG. 7, the first part (1) is the one with the smaller diameter than the second part (2) and has a flange like undercut region on its end distal to the needle side. The second part (2) has on its inner surface the slanted region. When the first part (1) is pushed past the slanted region of the second part (2), the flange like undercut locks behind the slanted region.

The second version is an adapter comprising a second part made of polypropylene using a screw on mechanism for the connection between the first and the second part. A cross-sectional view of such an adapter is shown in FIG. 8. In this embodiment, the first part (1) comprises an outer thread and the second part (2) comprises a corresponding inner thread for screwing the two parts together. The locking part (4) is in this case the thread.

For comparison, all of them have been fitted to the same type of standard Luer conical fitting syringe made of glass as described in ISO 11040-4:2015.

### a) Luer Cone Breakage Resistance Test (ISO 11040-4:2015, Annex C.2)

For reference purposes, the cone breaking force of a standard Luer conical fitting syringe made of glass has been determined with and without an adapter of the prior art installed in a Luer cone breakage resistance test according to ISO 11040-4:2015, Annex C.2 (point of force transfer: 2 mm from the tip of the syringe barrel). The statistics of the measurements are based on a set of 20 syringes tested in each case. Thereafter, the same type of syringes has been used with the respective adapters according to this disclosure and tested. The results are summarized in the table below.

| | Mean cone | | | |
|---|---|---|---|---|
| | breaking | Standard | | |
| Material/ | force | deviation | F_{Min} | F_{Max} |
| design | [N] | [N] | [N] | [N] |
| Reference | 171.0 | 47.2 | 63.3 | 253.0 |
| (glass body | | | | |
| without | | | | |
| adapter) | | | | |
| Reference | 56.68 | 12.88 | 30.9 | 80.6 |
| (with adapter, | | | | |
| no coating) | | | | |
| PA 11/click | 100.6 | 12.68 | 86.0 | 108.9 |
| mechanism¹ | | | | |
| PP/click | 99.5 | 12.00 | 82.6 | 107.4 |
| mechanism | | | | |
| PP/screw on | 89.92 | 10.88 | 74.10 | 106.10 |
| mechanism | | | | |

¹Cone breakage measurement with a setup according to ISO 11040-4: 2015, Annex C.2 (point of force transfer: 2 mm from the tip of the syringe barrel) results here in a bending of the polymer cone of the adapter. Hence, the measurements for PA 11 have been made with the point of force transfer moved to 5.5 mm from the tip of the syringe barrel. For the normal ISO 11040-4: 2015, Annex C.2 setup (with 2 mm distance) it can be stated that the cone breakage values are at least higher than the measured maximum for bending which has been 71.23N.

### b) Dye Solution Tightness Test (ISO 11040-4:2015, Annex H)

A known method for determining a tightness of a container is the so-called dye solution tightness test, which is laid out in Annex H to Part 4 of the international standard ISO 11040, 3rd edition issued 1 Apr. 2015. According to this standard, sub-assembled syringes which are filled with liquid and closed with a plunger stopper are submerged in a, normally aqueous, dye solution. After a depressurisation/re-pressurisation cycle, the sub-assembled syringes are inspected for leakage by checking the presence or absence of ingress of the dye solution into the syringe. To this end, the dye solution together with the syringes submerged therein is placed in a vacuum chamber, which produces and holds a negative pressure of at least 270 mbar during a period of half an hour. In the case that a leak is present in one of the containers, a portion of the content inside the container, which is still at atmospheric pressure, will be pressed out through the leak. Afterwards, when the vacuum chamber becomes re-pressurized, a portion of the dye solution is pressed into the container through the leak by the ambient pressure to compensate for the volume of the content that previously had been pressed out. Such dye can subsequently be detected using optical or spectroscopic techniques.

In an alternative method, a transfer of gas is detected which takes place during a depressurisation/re-pressurization cycle between air in a headspace of the container and a reference gas, for example, carbon dioxide, in the vacuum chamber. Since the reference gas is typically invisible, subsequent inspection of the container(s) is mostly performed by means of spectroscopy to thereby detect an anomalous gas composition or concentration of the reference gas in the headspace.

The result of the test is indicated by a pass or fail rating.

| Material/ | Test result |
|---|---|
| design | [pass/fail] |
| PA 11/click | pass |
| mechanism | |
| PP/click | pass |
| mechanism | |
| PP/screw on | pass |
| mechanism | |

### c) Setting Force Test (Analog to ISO 11040-4:2015, Annex H)

The setting force measured here is the force required to irreversibly connect the adapter by a click mechanism to the cone of the syringe barrel. The setting force may be determined analog to ISO 11040-4:2015, Annex H by pushing at a speed of 100 mm/min instead of pulling. For reference purposes, the setting force of an adapter of the prior art on a standard Luer conical fitting syringe made of glass has been determined.

| | Mean setting | Standard | | |
|---|---|---|---|---|
| Material/ | force | deviation | F_{Min} | F_{Max} |
| design | [N] | [N] | [N] | [N] |
| Reference | ca. 60 | — | — | — |
| PA 11/click | 57.92 | 8.67 | 38.07 | 73.42 |
| mechanism | | | | |
| PP/click | 54.62 | 8.11 | 35.71 | 71.55 |
| mechanism | | | | |

### d) Pull Off Force Test (ISO 11040-4:2015, Annex G.3)

With the pull off force test, the force required to pull the attached adapter from the cone of the syringe is measured. The pull off force may be determined according to ISO 11040-4:2015, Annex G.3. For reference purposes, the pull off force of an adapter of the prior art on a standard Luer conical fitting syringe made of glass has been determined.

| | Mean pull | Standard | | |
|---|---|---|---|---|
| Material/ | off force | deviation | F_{Min} | F_{Max} |
| design | [N] | [N] | [N] | [N] |
| Reference | 189.58 | 34.80 | 123.3 | 296.11 |
| PA 11/click | 99.1 | 10.78 | 70.2 | 108.35 |
| mechanism | | | | |
| PP/click | 95.7 | 9.84 | 68.9 | 103.5 |
| mechanism | | | | |
| PP/screw on | 144.98 | 11.5 | 118.2 | 171.7 |
| mechanism | | | | |

### e) Young's Modulus of the Materials (ISO 527-1/-2:2019)

The Young's modulus of the polymer materials used for the second part may be determined according to ISO 527-1/-2:2019.

| | Young's modulus |
|---|---|
| Material | [GPa] |
| Polyamide 11 | 1.5 |
| Polypropylene | 1.1 |

### Items of the Disclosure

The present disclosure is characterized by one or more of the following items.

Item 1. System for long-term storage of a pharmaceutical composition, comprising:
a syringe barrel, comprising:
   a front end comprising a cone, and
   a back end;
a plunger inserted into the back end; and
an adapter connected to the front end, comprising:
   a needle,
   an adapter body connecting the needle with the syringe barrel, and
   optionally a needle shield covering the needle;
wherein
   a pull-off force of the adapter is 1 N to 500 N, measured according to ISO 11040-4:2015, Annex G.3; and
   a cone breakage force is 1 N or more and/or 400 N or less, measured according to ISO 11040-4:2015, Annex C.2.

Item 2. System according to item 1, wherein
the pull-off force of the adapter is 50 N to 400 N, preferably 60 N to 300 N, more preferably 70 N to 250 N, more preferably 80 N to 200 N, more preferably 85 N to 150 N, measured according to ISO 11040-4:2015, Annex G.3; and/or
the cone breakage force is 5 N or more, preferably 20 N or more, more preferably 40 Nor more, more preferably 50 N or more, more preferably 60 N or more and/or 300 N or less, preferably 200 N or less, more preferably 150 N or less, measured according to ISO 11040-4:2015, Annex C.2.

Item 3. System according to item 1 or 2, wherein the system passes the container closure integrity test according to ISO 11040-4:2015, Annex H.

Item 4. System according to item 3, wherein the system passes the container closure integrity test according to ISO 11040-4:2015, Annex H after a storage time of 7 days, preferably 30 days, more preferably 100 days, more preferably 3 months, more preferably 6 months, more preferably 1 year, more preferably 2 years, more preferably 5 years at 15° C.-30° C. at ambient conditions or at 40° C.±2° C. at 75±5% relative humidity.

Item 5. System according to one of the preceding items, wherein
the syringe barrel comprises, preferably is made of, glass; and/or
the adapter body comprises polymer.

Item 6. System according to one of the preceding items, wherein the syringe barrel comprises:
a shoulder; and
wherein the cone comprises:
   a tapering region including the cone's broadest outer circumference, and
   an undercut having an outer circumference smaller than the cone's broadest outer circumference, wherein preferably the undercut is located between the tapering region and the shoulder of the syringe barrel.

Item 7. System according to one of the preceding items, wherein the adapter has an adapter rotation resistance force on the cone of 0.03 Nm to 1 Nm, preferably 0.04 Nm-0.6 Nm, preferably 0.05 Nm-0.4 Nm, preferably 0.06 Nm-0.3 Nm.

Item 8. System according to one of the preceding items, wherein the adapter is tilt-proof fitted to the syringe barrel so that a central axis of the needle is congruent with a central axis of the syringe barrel.

Item 9. System according to one of the preceding items, wherein the needle is mounted fixed or movable within the adapter body.

Item 10. System according to one of the preceding items, wherein the adapter body comprises:
a first part supporting the needle, and
a second part being in contact with the cone, preferably with the undercut of the cone.

Item 11. System according to item 10, wherein the first part and the second part are irreversibly connected, preferably by a click mechanism.

Item 12. System according to item 11, wherein the setting force of the second part of the adapter on the cone and/or the setting force of the adapter on the cone and/or the force to irreversibly connect the first part and the second part by a click mechanism to reach the click point of the click mechanism is 10 N to 300 N, preferably 20 N to 150 N, more preferably 50 N to 120 N, wherein the setting force is determined analog to ISO11040-4:2015, Annex H by pushing at a speed of 100 mm/min instead of pulling.

Item 13. System according to one of items 10 to 12, wherein the material of the second part comprises or consists of a polymer.

Item 14. System according to item 13, wherein the polymer is chosen from polypropylene (PP), polyethylene terephthalate (PET), or polyamide (PA) as well as their copolymers and blends containing at least 50 wt-% of said polymers.

Item 15. System according to one of items 13 or 14, wherein the polymer is PA 11.

Item 16. System according to one of items 13 to 15, wherein a deformation of the second part is elastic or elastic and plastic.

Item 17. System according to one of items 13 to 16, wherein the geometric design of the second part and/or the polymer for the second part are selected such that the maximal deformation of the second part when being slid over the cone onto the undercut of the syringe results in a strain ε of the polymer within a range of εₑₗ≤ε≤ε_{Y} or a range of εₑₗ≤ε≤εₘₐₓ for polymers without a yield point, wherein εₑₗ is the strain at the elastic limit, ε_{Y} is the strain at the yield point, and εₘₐₓ is the maximum strain, and/or the second part has at a position on the undercut of the cone a residual stress σᵣₑₛ from a remaining elastic deformation εᵣₑₛ.

Item 18. System according to one of items 13 to 17, wherein a ratio of the Vickers hardness of the syringe barrel material, when determined according to DIN EN ISO 6507-1:2018-07, to the shore D hardness of the material of the second part, when determined according to DIN ISO 7619-1:2012-02, is larger than 5.86.

Item 19. System according to one of items 10 to 18, wherein a sealing member is arranged between the first part and the syringe barrel.

Item 20. System according to item 19, wherein the sealing member is in contact with the cone, preferably the terminal part of the cone located at the distal side of the syringe barrel.

Item 21. System according to one of items 19 or 20, wherein the sealing member has a Shore A hardness, measured according to ASTM D2240:2021, 10 seconds, of 20 to 80, preferably 30 to 70, more preferably 45 to 65, more preferably 55 to 60.

Item 22. System according to one of items 19 to 21, wherein the sealing member is compressed by the click mechanism at least partially, preferably at least partially by 10% to 80%, preferably 20% to 70%, more preferably 30% to 60%, more preferably 40% to 50%.

Item 23. System according to one of items 19 to 22, wherein a Young's modulus of the sealing member is from 0.1 MPa to 5 MPa, preferably from 1 MPa to 4 MPa, more preferably from 1.5 MPa to 3 MPa, determined according to ISO 527-1/-2:2019.

Item 24. System according to one of items 19 to 23, wherein a thickness of the sealing member, preferably in its compressed state, is 0.05 mm to 3.00 mm, preferably 0.5 mm to 2.50 mm, preferably 0.80 mm to 2.20 mm.

Item 25. System according to one of items 19 to 24, wherein a material of the sealing member comprises, preferably consists of, a polymer, preferably an elastomer, more preferably a thermoplastic elastomer.

Item 26. System according to one of items 1 to 13, wherein the second part is a retaining part.

Item 27. System according to one of items 1 to 13 or 26, wherein the second part has essentially a ring shape which is not fully closed and/or has a gap and/or which can be widened in diameter.

Item 28. System according to one of items 1 to 13, 26 or 27, wherein the second part has essentially a ring shape which exerts a spring force in a direction of its central axis.

Item 29. System according to one of items 1 to 13 or 26 to 28, wherein a ratio of an inner circumference of the second part to the cone's broadest outer circumference is between 85% [mm/mm] and 99% [mm/mm] or between 90% [mm/mm] and 99% [mm/mm], when determined by measuring an inner diameter of the second part by means of a visual measurement device after disassembling it and elastic relaxation.

Item 30. System according to one of items 1 to 13 or 26 to 29, wherein a ratio of the inner circumference of the second part to the circumference of the undercut of the cone is from 90% [mm/mm] up to 107% [mm/mm], when determined by measuring the inner diameter of the second part by means of a visual measurement device after disassembling it and elastic relaxation.

Item 31. System according to one of items 1 to 13 or 26 to 30, wherein a ratio of a radial force of the second part to the pull-off force of the adapter is 1% to 20,000%[N/N], preferably 2% to 5,000% [N/N], more preferably 5% to 200% [N/N], more preferably 10% to 100% [N/N], more preferably 20% to 50% [N/N].

Item 32. System according to one of items 1 to 13 or 26 to 31, wherein the radial force of the second part is 5 N to 200 N, preferably 10 N to 180 N, more preferably 20 N to 150 N, more preferably 30 N to 120 N, more preferably 40 N to 100 N, more preferably 50 N to 80 N.

Item 33. System according to one of items 1 to 13, or 26 to 32, wherein the pull-off force of the adapter is the pull of force of the second part.

Item 34. System according to one of items 1 to 13 or 27 to 34, wherein the material of the second part comprises, preferably consists of, a metal, preferably a metal comprising iron and/or aluminum, more preferably stainless steel.

Item 35. System according to one of items 1 to 13 or 26 to 34, wherein a thickness of the second part is 0.03 mm to 1 mm, preferably 0.05 mm to 0.8 mm, more preferably 0.1 mm to 0.4 mm, more preferably 0.15 mm to 0.3 mm.

Item 36. System according to one of items 1 to 13 or 26 to 35, wherein a ratio of the Young's modulus [GPa], determined according to ISO 527-1/-2:2019, to the thickness of the second part [mm] is 50 to 10,000 [GPa/mm], preferably 100 to 8,000 [GPa/mm], preferably 200 to 5,000 [GPa/mm], preferably 300 to 2,000 [GPa/mm], preferably 500 to 1,000 [GPa/mm].

Item 37. System according to one of items 1 to 13 or 26 to 36, wherein the second part is completely surrounded by the first part and/or the second part is embedded in the first part.

Item 38. System according to one of items 1 to 13, wherein the cone and/or the second part comprise(s) at least one area which is coated by a single-layer or multi-layer coating.

Item 39. System according to item 38, wherein the at least one area comprises the tapering region having a broadest circumference of the cone and/or the undercut of the cone, preferably the undercut.

Item 40. System according to one of items 38 to 39, wherein the coating reduces surface defects on the cone and/or reduces the impact when the second part is clicked to the cone.

Item 41. System according to one of items 38 to 40, wherein the coating comprises a polymer, in particular an elastomer and/or a thermoplastic and/or a duromer and/or a silicone, and/or a ceramic.

Item 42. System according to one of items 38 to 41, wherein the coating has a thickness, preferably a mean thickness, of 40 nm to 200 μm, preferably 70 nm to 60 μm, preferably 80 nm to 50 μm, preferably 90 nm to 40 μm.

Item 43. System according to one of items 38 to 42, wherein a ratio of the pull-off force [N] to a thickness of the coating [nm] is 0.0004 to 8.75 [N/nm], preferably 0.0005 to 4 [N/nm], more preferably 0.0025 to 1 [N/nm], more preferably 0.01 to 0.1 [N/nm].

Item 44. System according to one of items 38 to 43, wherein a ratio of the hardness of the glass, expressed as the Young's Modulus in MPa, determined according to ISO 527-1/-2:2019, to the hardness of the coating, expressed as Young's Modulus in MPa, determined according to ISO 527-1/-2:2019, is 4 to 10,000 [MPa/MPa], preferably 10 to 1,000 [MPa/MPa], preferably 20 to 500 [MPa/MPa], preferably 40 to 200 [MPa/MPa], preferably 60 to 100 [MPa/MPa].

### REFERENCE NUMERALS

1 First part
2 Second Part
3 Sealing member zone
4 Locking part
5 Sealing member
6 Needle
7 Tapering region
8 Undercut
9 Shoulder
10 Tapering region coating area
11 Undercut coating area
12 Lug
13 Slot

1-44. (canceled)

45. A system for long-term storage of a pharmaceutical composition, comprising:
a syringe barrel including a front end comprising a cone, and a back end;
a plunger inserted into the back end; and
an adapter connected to the front end, the adapter including a needle, an adapter body connecting the needle with the syringe barrel,
wherein
a pull-off force of the adapter is 1 N to 500 N, measured according to ISO 11040-4:2015, Annex G.3; and
a cone breakage force is 1 N or more and/or 400 N or less, measured according to ISO 11040-4:2015, Annex C.2.

46. The system as recited in claim 45 wherein the pull-off force of the adapter is 50 N to 400 N; or the cone breakage force is 5 N or more.

47. The system as recited in claim 45 wherein the system passes a container closure integrity test according to ISO 11040-4:2015, Annex H.

48. The system as recited in claim 45 wherein the system passes a container closure integrity test according to ISO 11040-4:2015, Annex H after a storage time of 7 days, at 15° C.-30° C. at ambient conditions or at 40° C.±2° C. at 75±5% relative humidity.

49. The system as recited in claim 45 wherein the syringe barrel includes glass or the adapter body includes polymer.

50. The system as recited in claim 45 wherein the syringe barrel include a shoulder; and wherein the cone including:
a tapering region including a broadest outer circumference of the cone, and
an undercut having an outer circumference smaller than the broadest outer circumference.

51. The system as recited in claim 45 wherein the adapter has an adapter rotation resistance force on the cone of 0.03 Nm to 1 Nm.

52. The system as recited in claim 45 wherein the adapter is tilt-proof fitted to the syringe barrel so that a central axis of the needle is congruent with a central axis of the syringe barrel.

53. The system as recited in claim 45 wherein the needle is movable within the adapter body.

54. The system as recited in claim 45 wherein the adapter body includes a first part supporting the needle, and a second part being in contact with the cone.

55. The system as recited in claim 54 wherein the first part and the second part are irreversibly connected.

56. The system as recited in claim 54 wherein a setting force of the second part of the adapter on the cone or a setting force of the adapter on the cone or a force to irreversibly connect the first part and the second part by a click mechanism to reach a click point of the click mechanism is 10 N to 300 N, wherein the setting force is determined according to ISO11040-4:2015, Annex H by pushing at a speed of 100 mm/min instead of pulling.

57. The system as recited in claim 54 wherein a material of the second part includes a polymer.

58. The system as recited in claim 57 wherein the polymer is chosen from polypropylene (PP), polyethylene terephthalate (PET), or polyamide (PA) as well as their copolymers and blends containing at least 50 wt-% of such polymers.

59. The system as recited in claim 57 wherein the polymer is PA 11.

60. The system as recited in claim 57 wherein a deformation of the second part is elastic or elastic and plastic.

61. The system as recited in claim 57 wherein a geometric design of the second part is selected such that the maximal deformation of the second part when being slid over the cone onto the undercut of the syringe results in a strain ε of the polymer within a range of εₑₗ≤ε≤ε_{Y} or a range of εₑₗ≤ε≤εₘₐₓ for polymers without a yield point, wherein εₑₗ is the strain at the elastic limit, ε_{Y} is the strain at the yield point, and εₘₐₓ is the maximum strain, and/or the second part has at a position on the undercut of the cone a residual stress σᵣₑₛ from a remaining elastic deformation εᵣₑₛ.

62. The system as recited in claim 57 wherein a ratio of the Vickers hardness of a syringe barrel material, when determined according to DIN EN ISO 6507-1:2018-07, to the shore D hardness of the material of the second part, when determined according to DIN ISO 7619-1:2012-02, is larger than 5.86.

63. The system as recited in claim 54 further comprising a seal member arranged between the first part and the syringe barrel.

64. The system as recited in claim 63 wherein the seal member is in contact with the cone.

65. The system as recited in claim 63 wherein the seal member has a Shore A hardness, measured according to ASTM D2240:2021 of at least 10.

66. The system as recited in claim 63 wherein the seal member is compressed by the syringe barrel.

67. The system as recited in claim 63 wherein a Young's modulus of the seal member is from 0.1 MPa to 5 MPa, determined according to ISO 527-1/-2:2019.

68. The system as recited in claim 63 wherein a thickness of the seal member is 0.05 mm to 3.00 mm.

69. The system as recited in claim 63 wherein a material of the seal member includes a polymer.

70. The system as recited in claim 54 wherein the second part is a retaining part.

71. The system as recited in claim 54 wherein the second part has a ring shape not fully closed or a gap or widened in diameter.

72. The system as recited in claim 54 wherein the second part has a ring shape exerting a spring force in a direction of a central axis.

73. The system as recited in claim 54 wherein a ratio of an inner circumference of the second part to a broadest outer circumference if the cone is between 85% [mm/mm] and 99% [mm/mm], when determined by measuring an inner diameter of the second part using a visual measurement device after disassembling the system and elastic relaxation.

74. The system as recited in claim 54 wherein a ratio of an inner circumference of the second part to a circumference of the undercut of the cone is from 90% [mm/mm] up to 107% [mm/mm], when determined by measuring an inner diameter of the second part by means of a visual measurement device after disassembling the system and elastic relaxation.

75. The system as recited in claim 54 wherein a ratio of a radial force of the second part to the pull-off force of the adapter is 1% to 20,000% [N/N].

76. The system as recited in claim 54 wherein a radial force of the second part is 5 N to 200 N.

77. The system as recited in claim 54 wherein a pull-off force of the adapter is a pull-off force of the second part.

78. The system as recited in claim 54 wherein a material of the second part includes a metal.

79. The system as recited in claim 54 wherein a thickness of the second part is 0.03 mm to 1 mm.

80. The system as recited in claim 54 wherein a ratio of the Young's modulus [GPa], determined according to ISO 527-1/-2:2019, to the thickness of the second part [mm] is 50 to 10,000 [GPa/mm].

81. The system as recited in claim 54 wherein the second part is completely surrounded by the first part or the second part is embedded in the first part.

82. The system as recited in claim 54 wherein the cone or the second part includes at least one area which is coated by a coating.

83. The system as recited in claim 82 wherein the at least one area includes a tapering region having a broadest circumference of the cone or the undercut of the cone.

84. The system as recited in claim 82 wherein the coating reduces surface defects on the cone or reduces the impact when the second part is connected to the cone.

85. The system as recited in claim 82 wherein the coating includes a polymer or a ceramic.

86. The system as recited in claim 82 wherein the coating has a thickness of 40 nm to 200 μm.

87. The system as recited in claim 82 wherein a ratio of a pull-off force [N] to a thickness of the coating [nm] is 0.0004 to 8.75 [N/nm].

88. The system as recited in claim 82 wherein a ratio of the hardness of the glass, expressed as the Young's Modulus in MPa, determined according to ISO 527-1/-2:2019, to the hardness of the coating, expressed as Young's Modulus in MPa, determined according to ISO 527-1/-2:2019, is 4 to 10,000 [MPa/MPa].

89. The system as recited in claim 45 further comprising a needle shield covering the needle.
